(19) United States
(12) Patent Application Publication
(10) Pub. No.: US 2011/0171145 A1
(43) Pub. Date: Jul. 14, 2011

(54) Tooth whitener and stain remover

(76) Inventor: Garrett Caldwell, Alameda, CA (US)

(21) Appl. No.: 13/005,982

(22) Filed: Jan. 13, 2011

Related U.S. Application Data
(60) Provisional application No. 61/294,914, filed on Jan. 14, 2010.

(51) Int. Cl. A61K 8/38; A61Q 11/00

(52) U.S. Cl. 424/53

(57) ABSTRACT

An inventive method resulting in a tooth whitening composition which solves daily regular facial and interproximal tooth staining and long term tooth color degradation; freshens breath due to minor halitosis; creates the appearance of brighter teeth, by way of its unique formulation, without changing tooth shade establishing tooth surface light refraction allowing the user with stained teeth to achieve immediate appearance of a brighter smile while the oxidizing formulation removes enamel staining and lightens the shade of the tooth enamel with regular use; gives user who may be sensitive to bleaching systems containing high levels of hydrogen peroxide a low dose alternative to prevent enamel and dentin tubual sensitivity; allows patients who have received prescription level bleaching at a dental office to regularly maintain the shade of their dentition for longer periods between retreatment at a dental office; and dispensed in a unique, convenient delivery system.

### REFERENCES CITED

U.S. Pat. No. 7,846,411

U.S. Pat. No. 7,094,393

U.S. Pat. No. 6,231,343

U.S. Pat. No. 7,621,746

U.S. Pat. No. 7,172,423

U.S. Pat. No. 7,785,572

U.S. Pat. No. 7,335,025

U.S. Pat. No. 6,361,320

U.S. Pat. No. 7,802,988

U.S. Pat. No. 7,201,578

### BACKGROUND OF THE INVENTION

The present invention relates to a tooth whitener, stain remover with a unique, light refraction formulation.

### DESCRIPTION OF CONVENTIONAL ART

Tooth whitening is considered one of the fastest growing cosmetic segments. It has grown remarkably as the demand for whiter, brighter teeth has increased by men and women. The most common method is generally one using a hydrogen peroxide solution in either an aqueous base or a base of alcohol.

The existing methods using hydrogen peroxide and/or other base ingredient solutions such as Calcium Peroxyphospate as described in U.S. Pat. No. 7,846,411 or U.S. Pat. No. 7,094,393 are based on the common idea in which the solution used creates the desired response quickly but at a high cost to the user. To achieve the desired results quickly existing systems must utilize higher levels of hydrogen peroxide, above 10% hydrogen peroxide, which may cause dentin tubual sensitivity upon application. Also, existing systems containing higher levels, above 10% hydrogen peroxide, may cause the blanching of tissues surrounding the teeth upon application. Existing methods rely solely on the use of higher doses of hydrogen peroxide to gain the desired results.

Existing methods for whitening teeth using titanium dioxide as a base ingredient rely on irradiating the formula with light to activate the whitening process as in U.S. Pat. No. 6,231,343.

Existing methods for dispensing tooth whitening products is also a key part of the inventive process. Tooth whitening products are generally dispensed by placing trays in the mouth filled with tooth whitening solutions as described by U.S. Pat. Nos. 7,621,746 and 7,172,423. Other products are dispensed by placing strips saturated with the whitening product on the teeth as described in U.S. Pat. No. 7,785,572 or U.S. Pat. No. 7,335,025. And still other whitening solutions are placed on the teeth in a dental office and irradiated with light as shown in U.S. Pat. Nos. 6,361,320, 7,802,988, and 7,201,578. All systems purport to work however inconvenience, dentin tubual sensitivity, blanching of tissues, and control play into the choice. As such, studies performed by our team show the application of lip sticks and lip gloss to be a strong social phenomenon. By using a tube with a brush applicator as a delivery system to allow the user to brush the whitening product onto their teeth in a motion similar to using a paint brush, we believe is a unique concept, adapts to the existing social modality.

### DESCRIPTION OF INVENTION

The present invention utilizes technology that is cosmetically based rather than solely chemically based, to whiten and brighten dentition. Current methods contain high doses of hydrogen peroxide in an alcohol base or utilize high levels, above 10% hydrogen peroxide, which may cause dentin tubual sensitivity upon application. Current methods contain higher levels, above 10% hydrogen peroxide, may cause blanching of tissues surrounding teeth upon application. The present invention relies on the unique light refracting properties of its ingredients rather than solely on the use of higher levels of hydrogen peroxide whether the hydrogen peroxide contained in the current device or systems are hydrogen peroxide or PEROXYDONE® PEROXYDONE® is a registered trademark of International Specialty Corporation (ISP).

As such, an objective of the present invention is to make a bleaching and whitening product by which the user who may be sensitive to bleaching systems containing high levels of hydrogen peroxide a low dose alternative to prevent enamel and dentin tubual sensitivity. The inventive process maintains, as in the objectives, that by having lower hydrogen peroxide levels it avoids the potential for blanching of tissues surrounding teeth upon application. In particular, the unique aspect of the present invention is contained in the use of low dose hydrogen peroxide to achieve whiter teeth over a period of time rather than by high levels of hydrogen peroxide which may cause sensitivity as described above. At a level of between 3.33 to 3.68% hydrogen peroxide per dose, in the present invention, the whitener may be used up to three times per day thus preventing enamel and dentin tubual sensitivity as well as avoiding the potential for blanching of tissues surrounding teeth upon application.

A fully unique aspect of the present invention is the use of titanium dioxide and mica within the formulation. By relying on the light refraction technology, the invention creates the appearance of brighter teeth by reflecting light without changing the tooth shade by establishing tooth surface light refraction allowing a user with stained teeth to achieve the immediate appearance of a brighter smile.

Titanium dioxide has a high refractive index surpassed only by diamonds, which is defined as a measure of the speed of light in that substance. The refractive index is expressed as a ratio of the speed of light in vacuum relative to that in the considered medium. The velocity at which light travels in vacuum is a physical constant, and the fastest speed at which energy or information can be transferred. Titanium dioxide contains an optical properties in two phases Anatase with a refractive index of 2.49, density (g.cm⁻³) 3.84 and has a crystal structure which is tetragonal and Ruile with a the refractive index is 2.903, density (g.cm⁻³) 4.26, and tetragonal crystal structure. This translates into use within the present invention in its unique formulation to refract light quickly and create an impression of whiter, brighter teeth. As such, the invention relies on light refracting properties of its ingredients rather than solely on the use of higher levels of hydrogen peroxide whether the hydrogen peroxide contained in the current device or systems are hydrogen peroxide or PEROXYDONE®.

The use of TIMERON M 45 GLEAMER FLAKE® (a trademark of Merck Chemicals), which contains both titanium dioxide and mica, and eliminating regular hydrogen peroxide from the formula and replacing it using PEROXYDONE K30® and PEROXYDONE K90® as well as eliminating all alcohol from the formulation, the product is able to achieve unique lightening, utilizing refracting characteristics creating both immediate as well as long term whitening through its oxidizing formulation as described above.

In addition to standard applications, after consuming stain causing foods and/or drinks (such as coffee, tea, red wine), the oxidizer contained within the formulation will at its present level provide immediate removal of surface staining In addition, the sparkle effect of the mica, contained within the TIMERON® M 45 GLEAMER FLAKE®, creates the appearance of brighter teeth without an actual change of existing tooth shade which may be a lower value caused by long term staining, through a unique formulation of ingredients establishing tooth surface light refraction allowing the user with stained teeth to achieve immediate appearance of a brighter smile.

An additional objective of the invention is to provide relief for minor halitosis by using natural flavorings and sweeteners. There is also a future potential for creating a mouthwash/rinse of lower viscosity then traditional mouthwashes.

Additionally, our research showed an objective of the invention was delivery. Our research focused on determining how an individual may use the invention in a natural and convenient way. The lipstick concept, as described above in Conventional Art, expanded the search for a distinctly unique way to apply the tooth whitening composition onto the teeth in a portable easy to use way. As such, the use of a lipgloss style applicator with a brush tip was determined to be the ideal solution for delivery. The brush tip allows the user to apply the invention directly to all surfaces of the teeth by painting or brushing it on. As such it was determined a dial up applicator should be used. The preferred applicator consists of a brush head made of nylon and polypropylene and measures 1.35 inches in length and 0.3 inches in diameter; an applicator tube made of ethylene vinyl acetate, polypropylene, and POM and is 4.5 inches in length and 0.6 inches in diameter; and a cap made of polypropylene and is 1.8 inches in length and 0.6 inches in diameter. Although the current invention prefers the applicator as described, additional variations of the applicator are contemplated by the invention.

### SUMMARY OF THE INVENTION

The inventive method presents a tooth whitener in a gel form, which includes a tooth whitener having a concentration of hydrogen peroxide between 3.33 to 3.68% and includes Food Grade Glycerin, PEROXYDONE K-90®, PEROXYDONE K-30®, PLASADONE K-90®, flavoring, Monoammonium Glycyrrhizinate, TIMERON M 45 GLEAMER FLAKE®, and a balance of water dispensed in a unique delivery system consisting of a brush head made of polypropylene and nylon; a dial-up applicator tube made of ethylene vinyl acetate and polypropylene, and POM; and a cap made of polypropylene

### DISCLOSURE OF THE INVENTION

The preferred embodiment of the invention is described in further detail below, but the present invention is not in any way limited by the description:

The tooth whitening composition is comprised by mixing well 49.035% RO or distilled water by weight and 27% by weight of Glycerin, food grade 99.7%:
a. Glycerin is a clear, colorless, odorless, viscous liquid that is widely used in pharmaceutical, personal care, and industrial applications. It it non-toxic, water soluble, has a neutral pH, non-flammable and is readily biodegradable. Food grade Glycerin is closely regulated by the FDA. It must meet stringent requirements of the United States Pharmacopeia (USP) and the Food Chemicals Codex (FCC)

Added to the composition is 16.65% by weight of a vinyl pyrrolidone polymers and hydrogen peroxide is added to the mixture, preferred use in the invention is PEROXYDONE K-90® and 4.97% of pharmaceutical grade polyvinylpyrrolidone with hydrogen peroxide preferably PEROXYDONE K-30® and 1.39% of a water-soluble polyvinylpyrrolidone preferably PLASDONE K-90® (a registered trademark of International Specialty Products).
a. A stable complex of vinyl pyrrolidone polymers and hydrogen peroxide, white, free-flowing powder. The properties of PEROXYDONE® releases hydrogen peroxide on contact with water; good film former and bioahesive; gorms clear, and nontacy and continuous films. PEROXYDONE® is soluble in water, ethanol, glycerol, various glycols and PEG 400. PEROXYDONE K-90® contains 16-20% hydrogen peroxide or H2O2 and PEROXYDONE K-30® contains 17-20% hydrogen peroxide or H2O2.
b. Other hydrogen peroxide generating ingredients are available, however, the preferred use for the invention is PEROXYDONE K-90® and PEROXYDONE K-30® as they offer a more concentrated and stable complex of hydrogen peroxide for optimum whitening results and retains stability without sacrificing strength or concentration.
c. PLASDONE K-90® is a linear homopolymers of vinylpyrrolidone and is a white or creamy white powders. It is soluble in water and most pharmaceutically acceptable solvents, physiologically inert, viscosity modifier in aqueous systems, film former, functions as a superior wet granulation tablet binder, improves solubility and enhances bioavailability of drug actives, provides viscosity modification, crystal inhibition and drug solubilization in liquid dosage forms, makes an excellent cohesive agent, stabilizer and protective colloid, aids in pore formation in membrane production, forms water-soluble bioadhesives for use with medical electrodes, and forms films for anti-coagulant coatings used in blood collection.

Added to the mixture of said ingredients in the tooth whitening composition is a flavor enhancer as such 0.75% of peppermint oil. The tooth whitening composition contains one or more flavoring agents. It may contain essential oils, natural or synthetic flavors, or mixtures thereof including but not limited to oils derived from plants and fruits such as citrus oils, fruit essences, mint, peppermint oil, spearmint oil, clove oil, oil of wintergreen, anise, sassafras, sage, cinnamon, lemon, orange, banana, cherry, apple, pineapple, grape, strawberry, blueberry, watermelon, papaya, and the like. These as natural or artificial flavoring agents may be used independently or combined in any blend acceptable to the senses. All such flavors and flavor blends are contemplated by the invention.
a. Peppermint Oil Redistilled from Ungerer, in the current invention, is an essential oil derived from a botanical (fruit, leaf, root, stem and/or flower) via cold process or steam distillation. Pure grade essential oils are 100% natural.

Added to the tooth whitening composition is 0.055% Monoammonium Glycyrrhizinate (MAG) to mask flavors and sweeten the composition preferably in the form of MAGNASWEET 100® (a registered trademark of Mafco). As such other sweetening and/or flavoring agents, to provide greater consumer appeal, are contemplated by the invention including but not limited to sucrose, maltose, dextrin, dried inverted sugar, fructose, levulose, galactose, corn syrup, sorbitol, mannitol, xylitol and the line.
a. The MAGNASWEET 100® series, as present in the current invention, is a line of products based on the traditional Monoammonium Glycyrrhizinate (MAG) which is the ammonia salt form of Glycyrrhizic Acid. Glycyrrhizic Acid is the unique ingredient found in licorice root. MAG is natural and has no licorice flavor. MAGNASWEET 100® has four key performance attributes: masking or eliminating aftertastes, intensifying sweetness, extending sweetness and enhancing other flavors. These attributes are what make the MAGNASWEET 100® series such a versatile ingredient in a wide variety of applications for beverages, foods, pharmaceuticals, nutraceuticals and cosmetics.

Added to the tooth whitening composition is 0.15% of a mixture of titanium dioxide and mica all preferably TIMERON M45 GLEAMER FLAKE®, although other such mixtures are contemplated by the invention.
a. TIMERON M45 GLEAMER FLAKE® is a mica coated with titanium dioxide. It contains a particle size (80%, microns): 20.0 110.0; D-50 (microns): 49.0 57.0; and is characterized as a silvery-white sparkling powder.

The resulting tooth whitening composition as defined by the present invention contains a low dose hydrogen peroxide level of 3.33 to 3.68%, a specific gravity of @25° C. (Grease Pyconmeter) 1.16-1.18, a pH@25° C. of 4.00-4.40, a viscosity RVT D/5 @25° C. (for one minute) of 2,000-2,500 cps (The viscosity of the composition is a direct correlation to provide a reasonable amount of time for the composition to remain on the teeth), and an appearance of a moderately viscous gel with a sparkle effect.

Once the tooth whitening composition as defined in the present invention is complete it is placed in a unique dispensing system. The preferred applicator consists of a brush head made of nylon and polypropylene and measures 1.35 inches in length and 0.3 inches in diameter; an applicator tube made of ethylene vinyl acetate, polypropylene, and POM and is 4.5 inches in length and 0.6 inches in diameter; and a cap made of polypropylene and is 1.8 inches in length and 0.6 inches in diameter. Although the current invention prefers the applicator as described, additional variations of the applicator are contemplated by the invention.

What is claimed:

1. A tooth whitening composition for contacting a tooth in an oral cavity, comprising of hydrogen peroxide, a thickening agent, a flavor enhancer, a sweetening/flavor masking agent, titanium dioxide and mica, and a balance of RO water or distilled water, wherein the resulting composition has: a) a hydrogen peroxide level between 3.33 to 3.68% per dose; b) a pH level of pH@25° C. of 4.00-4.40; c) a viscosity RVT D/5 @25° C. (for one minute) of 2,000-2,500 cps; and d) a specific gravity of specific gravity of @25° C. (Grease Pycnometer) 1.16-1.18.

2. A tooth whitening composition as defined in claim 1, wherein the content of the composition contains at least 49.035% RO water or distilled water.

3. A tooth whitening composition as defined in claim 1, wherein the content of the composition contains 27% by weight Glycerin, food grade 99.7%.

4. A tooth whitening composition as defined in claim 1, wherein the content of the composition contains 16.65% by weight of a stable complex of vinyl pyrrolidone polymers and hydrogen peroxide preferably PEROXYDONE K-90®.

5. A tooth whitening composition as defined in claim 1, wherein the content of the composition contains 4.97% by weight of a stable complex of vinyl pyrrolidone polymers and hydrogen peroxide preferably PEROXYDONE K-30®.

6. A tooth whitening composition as defined in claim 1, wherein the content of the composition contains 1.39% a linear homopolymers of vinylpyrrolidone preferably PLASDONE K-90®.

7. A tooth whitening composition as defined in claim 1, wherein the content of the composition contains a flavor enhancer, in current invention 0.75% by weight of peppermint oil, as such the tooth whitening composition may contain one or more flavoring agents, essential oils, natural or synthetic flavors, or mixtures thereof including but not limited to oils derived from plants and fruits such as citrus oils, fruit essences, mint, peppermint oil, spearmint oil, clove oil, oil of wintergreen, anise, sassafras, sage, cinnamon, lemon, orange, banana, cherry, apple, pineapple, grape, strawberry, blueberry, watermelon, papaya, and the like, therefore all such flavors and flavor blends are contemplated by the invention.

8. A tooth whitening composition as defined in claim 1, wherein the content of the composition contains 0.055% by weight of Monoammonium Glycyrrhizinate to mask and sweeten the flavor of the composition, as such the invention considers other options for masking and sweetening the formula such as sucrose, maltose, dextrin, dried inverted sugar, fructose, levulose, galactose, corn syrup, sorbitol, mannitol, xylitol and the like.

9. A tooth whitening composition as defined in claim 1, wherein the content of the composition contains a mixture of 0.15% by weight mixture of titanium dioxide and mica, preferably TIMERON M45 GLEAMER FLAKE®.

10. A tooth whitening composition as defined in claim 1, wherein the resulting composition can be is dispensed from a component consisting of a brush head made of polypropylene and nylon; a dial-up applicator tube made of ethylene vinyl acetate and polypropylene, and POM; and a cap made of polypropylene wherein additional variations of plastic dispensers are considered by the invention.
